(19) United States
(12) Patent Application Publication
(10) Pub. No.: US 2008/0188540 A1
(43) Pub. Date: Aug. 7, 2008

(54) PHARMACEUTICAL COMPOSITIONS CONTAINING SUBSTITUTED INDOLE ACID DERIVATIVES AS INHIBITORS OF PLASMINOGEN ACTIVATOR INHIBITOR-1 (PAI-1)

(75) Inventors: Marc Sadler Tesconi, Monroe, NY (US); Mannching Sherry Ku, Thiells, NY (US); Yan Xu, New City, NY (US); Hsueh-Ling Wu, Edison, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/026,300

(22) Filed: Feb. 5, 2008

Related U.S. Application Data
(60) Provisional application No. 60/899,491, filed on Feb. 5, 2007.

(51) Int. Cl. A61K 31/40; A61P 25/28

(52) U.S. Cl. 514/412

(57) ABSTRACT

Pharmaceutical compositions containing compounds of formula I are provided:

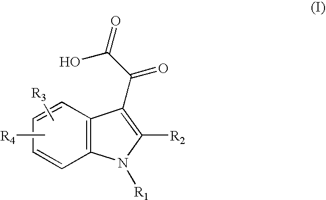

wherein the constituent variables are as defined herein. The compositions are useful as inhibitors of plasminogen activator inhibitor-1 (PAI-1) for treating conditions resulting from fibrinolytic disorders, such as deep vein thrombosis and coronary heart disease, Alzheimer's disease and pulmonary fibrosis.

### CROSS-REFERENCE TO RELATED APPLICATION

This application claims priority benefit of U.S. Provisional Application Ser. No. 60/899,491 filed Feb. 5, 2007, which is incorporated herein by reference in its entirety.

### FIELD OF THE INVENTION

This invention relates to pharmaceutical formulations containing substituted indole acid derivatives as inhibitors of plasminogen activator inhibitor-1 (PAI-1) useful for the treatment of a wide variety of conditions including deep vein thrombosis, coronary heart disease, pulmonary fibrosis, cognition impairment, senility and Alzheimer's disease.

### BACKGROUND OF INVENTION

Plasminogen activator inhibitor-1 (PAI-1) is a major regulatory component of the plasminogen-plasmin system. PAI-1 is the principal physiologic inhibitor of both tissue type plasminogen activator (tPA) and urokinase type plasminogen activator (uPA). Elevated plasma levels of PAI-1 have been associated with thrombotic events as indicated by animal experiments (Krishnamurti, Blood, 69, 798 (1987); Reilly, Arteriosclerosis and Thrombosis, 11, 1276 (1991); Carmeliet, Journal of Clinical Investigations, 92, 2756 (1993)) and clinical studies (Rocha, Fibrinolysis, 8, 294, 1994; Aznar, Haemostasis 24, 243 (1994)). Antibody neutralization of PAI-1 activity resulted in promotion of endogenous thrombolysis and reperfusion (Biemond, Circulation, 91, 1175 (1995); Levi, Circulation 85, 305, (1992)). Elevated levels of PAI-1 have also been implicated in diseases of women such as polycystic ovary syndrome (Nordt, Journal of Clinical Endocrinology and Metabolism, 85, 4, 1563 (2000)) and bone loss induced by estrogen deficiency (Daci, Journal of Bone and Mineral Research, 15, 8, 1510 (2000)). Accordingly, agents that inhibit PAI-1 would be of utility in treating conditions originating from fibrinolytic disorder such as deep vein thrombosis, coronary heart disease, pulmonary fibrosis, polycystic ovary syndrome, etc.

PAI-1 inhibitors, by virtue of their ability to lead to the activation of plasmin, are predicted to reduce the levels of both soluble and aggregated forms of Aβ40/42 peptide by enhanced proteolytic clearance. Since Aβ40/42 comprise amyloid plaques associated with Alzheimer's disease, use of the novel formulations of this invention are promising candidate treatments for the prevention/treatment of Alzheimer's disease.

The present invention describes pharmaceutical formulations containing certain indole-containing, PAI-1 inhibitors for use in treating various conditions where PAI-1 inhibition is desirable.

### SUMMARY OF THE INVENTION

This invention relates to pharmaceutical compositions containing compounds of formula (I), or a pharmaceutically acceptable salt, solvate or ester thereof:

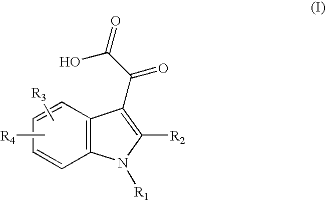

wherein:

R₁ is selected from C₁-C₈ alkyl, (—CH₂)ₙ—C₃-C₆ cycloalkyl, wherein n is an integer of from 0 to 3, pyridinyl, —CH₂-pyridinyl, phenyl or benzyl, the rings of the cycloalkyl, pyridinyl, phenyl and benzyl groups being optionally substituted by, from 1 to 3 groups selected from, halogen, C₁-C₄ alkyl, C₁-C₃ perfluoroalkyl, —O—C₁-C₃ perfluoroalkyl, C₁-C₃ alkoxy, —OH, —NH₂, or —NO₂;

R₂ is selected from H, C₁-C₆ alkyl, C₃-C₆ cycloalkyl, —CH₂—C₃-C₆ cycloalkyl, or C₁-C₃ perfluoroalkyl, —CH₂OH or CH₂OAc;

R₃ is selected from H, halogen, C₁-C₆ alkyl, C₁-C₃ perfluoroalkyl, C₁-C₆ alkoxy, C₃-C₆ cycloalkyl, —CH₂—C₃-C₆ cycloalkyl, C₄-C₆ cycloalkenyl, —CH₂—C₄-C₆ cycloalkenyl, —NH₂, or —NO₂; and

R₄ is phenyl substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, —NO₂ or (CO)C₁-C₆ alkyl.

In some embodiment, the composition comprises a liquid or emulsion. In some further embodiments, said liquid or emulsion comprises one or more solubilizers or emulsifiers, for example, 1, 2, 3, 4, or more solubilizers or emulsifiers.

### BRIEF DESCRIPTION FOR THE DRAWINGS

FIG. 1 Depicts the pH-solubility profile of [1-(4-tert-butylbenzyl)-5-(3-methylphenyl)-1H-indol-3-yl](oxo)acetic acid.

FIG. 2 Depicts the plasma concentration of [1-(4-tert-butylbenzyl)-5-(3-methylphenyl)-1H-indol-3-yl](oxo)acetic acid over 30 hours following single oral dose of the compound in a Cremophor based liquid formulation

### DETAILED DESCRIPTION OF THE INVENTION

The present teachings relate to pharmaceutical compositions containing compounds of formula (I), or a pharmaceutically acceptable salt, solvate or ester thereof: compound of formula (I), or a pharmaceutically acceptable salt or solvate thereof)

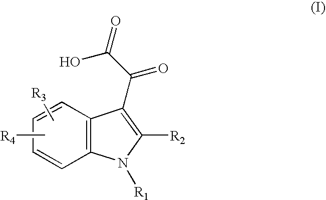

wherein:
R₁ is selected from C₁-C₈ alkyl, (—CH₂)ₙ—C₃-C₆ cycloalkyl, wherein n is an integer of from 0 to 3, pyridinyl, —CH₂-pyridinyl, phenyl or benzyl, the rings of the cycloalkyl, pyridinyl, phenyl and benzyl groups being optionally substituted by, from 1 to 3 groups selected from, halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is selected from H, C₁-C₆ alkyl, C₃-C₆ cycloalkyl, —CH₂—C₃-C₆ cycloalkyl, or C₁-C₃ perfluoroalkyl, —CH₂OH or CH₂OAc;
R₃ is selected from H, halogen, C₁-C₆ alkyl, C₁-C₃ perfluoroalkyl, C₁-C₆ alkoxy, C₃-C₆ cycloalkyl, —CH₂—C₃-C₆ cycloalkyl, C₄-C₆ cycloalkenyl, —CH₂—C₄-C₆ cycloalkenyl, —NH₂, or —NO₂; and
R₄ is phenyl substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, —NO₂ or (CO)C₁-C₆ alkyl.

In some embodiments, the composition comprises a liquid or emulsion. In some further embodiments, said liquid or emulsion comprises one or more solubilizers or emulsifiers, for example, 1, 2, 3, 4, or more solubilizers or emulsifiers

In some embodiments of this invention, said compound of formula (I) is a compound of formula (II) or formula (III), or a pharmaceutically acceptable salt, solvate or ester thereof:

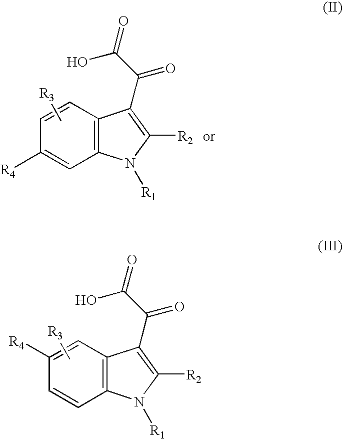

wherein:

R₁, R₂, R₃, and R₄ are as defined for previously in formula (I).

In some embodiments of this invention, the compound of formula (I) is a compound of formula (IV) or formula (V), or a pharmaceutically acceptable salt, solvate or ester thereof:

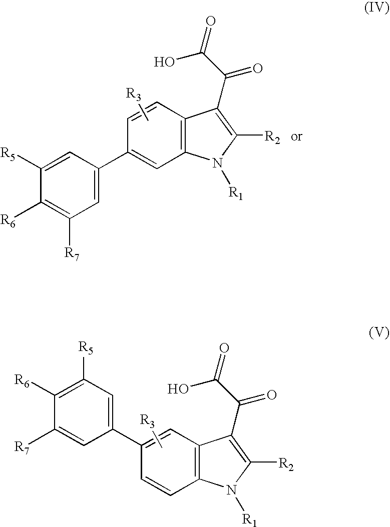

wherein:
R₁ is selected from C₁-C₈ alkyl, C₃-C₆ cycloalkyl, —CH₂—C₃-C₆ cycloalkyl, or benzyl, the rings of the cycloalkyl and benzyl groups being optionally substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is selected from H, C₁-C₆ alkyl, C₃-C₆ cycloalkyl, —CH₂—C₃-C₆ cycloalkyl, or C₁-C₃ perfluoroalkyl;
R₃ is selected from H, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂; and
R₅, R₆ and R₇ are independently selected from H, halogen, C₁-C₃ alkyl, C₁-C₃ perfluoroalkyl, —O—C₁-C₃ perfluoroalkyl, C₁-C₃ alkoxy, —OH, —NH₂, or —NO₂, provided that at least one of R₅, R₆ and R₇ is not H.

In some embodiments of this invention, the compound of formula (I) is a compound of formula (VI), or a pharmaceutically acceptable salt, solvate or ester thereof:

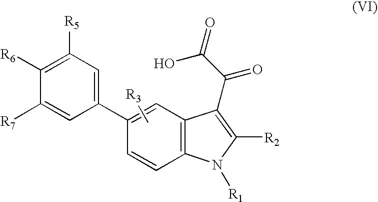

wherein:

R₁ is selected from benzyl, the benzyl group being optionally substituted by from 1 to 3 groups selected from halogen, C₁-C₄ alkyl, C₁-C₃ perfluoroalkyl, —O—C₁-C₃ perfluoroalkyl, or C₁-C₃ alkoxy;

R₂ is H;

R₃ is H; and

R₅, R₆ and R₇ are independently selected from H, halogen, C₁-C₃ alkyl, C₁-C₃ perfluoroalkyl, —O—C₁-C₃ perfluoroalkyl and C₁-C₃ alkoxy, provided that at least one of R₅, R₆ and R₇ is not H.

In some embodiments of this invention, the compound of formula (I) is
{1-Methyl-6-[4-(trifluoromethoxy)phenyl]-1H-indol-3-yl}(oxo)acetic acid;
{1-Methyl-6-[4-(trifluoromethyl)phenyl]-1H-indol-3-yl}(oxo)acetic acid;
{1-Ethyl-6-[4-(trifluoromethoxy)phenyl]-1H-indol-3-yl}(oxo)acetic acid;
{1-Ethyl-6-[4-(trifluoromethyl)phenyl]-1H-indol-3-yl}(oxo)acetic acid;
{1-Benzyl-6-[4-(trifluoromethoxy)phenyl]-1H-indol-3-yl}(oxo)acetic acid;
{1-Benzyl-6-[4-(trifluoromethyl)phenyl]-1H-indol-3-yl}(oxo)acetic acid;
{1-[4-(tert-Butyl)benzyl]-6-[4-(trifluoromethyl)phenyl]-1H-indol-3-yl}(oxo)acetic acid;
{1-[4-(tert-Butyl)benzyl]-6-[4-(trifluoromethoxy)phenyl]-1H-indol-3-yl}(oxo)acetic acid;
{1-Benzyl-5-[4-(trifluoromethyl)phenyl]-1H-indol-3-yl}(oxo)acetic acid;
{6-[4-(tert-Butyl)phenyl]-1-methyl-1H-indol-3-yl}(oxo)acetic acid;
[5-(4-Acetylphenyl)-1-benzyl-1H-indol-3-yl](oxo)acetic acid;
{1-Benzyl-5-[4-(trifluoromethoxy)phenyl]-1H-indol-3-yl}(oxo)acetic acid;
{1-Benzyl-4-[4-(trifluoromethyl)phenyl]-1H-indol-3-yl}(oxo)acetic acid;
{1-Benzyl-5-[4-(tert-butyl)phenyl]-1H-indol-3-yl}(oxo)acetic acid;
[1-Benzyl-5-(3-chloro-4-fluorophenyl)-1H-indol-3-yl](oxo)acetic acid;
{1-Benzyl-5-[3,5-bis(trifluoromethyl)phenyl]-1H-indol-3-yl}(oxo)acetic acid;
{1-Benzyl-7-[4-(trifluoromethoxy)phenyl]-1H-indol-3-yl}(oxo)acetic acid;
[1-Benzyl-7-(3-chloro-4-fluorophenyl)-1H-indol-3-yl](oxo)acetic acid;
{1-(4-tert-Butylbenzyl)-5-[4-(trifluoromethoxy)phenyl]-1H-indol-3-yl}(oxo)acetic acid;
{1-Benzyl-4-[4-(trifluoromethoxy)phenyl]-1H-indol-3-yl}(oxo)acetic acid;
[1-Benzyl-6-(3-chlorophenyl)-1H-indol-3-yl](oxo)acetic acid;
{1-Benzyl-5-[3-(trifluoromethoxy)phenyl]-1H-indol-3-yl}(oxo)acetic acid;
{1-(4-Methylbenzyl)-5-[4-(trifluoromethoxy)phenyl]-1H-indol-3-yl}(oxo)acetic acid;
{1-(4-Fluorobenzyl)-5-[4-(trifluoromethoxy)phenyl]-1H-indol-3-yl}(oxo)acetic acid;
[1-Butyl-5-(4-chlorophenyl)-1H-indol-3-yl](oxo)acetic acid;
[1-Butyl-5-(3-chlorophenyl)-1H-indol-3-yl](oxo)acetic acid;
[1-Butyl-5-(3-methoxyphenyl)-1H-indol-3-yl](oxo)acetic acid;
[1-Butyl-5-(4-methoxyphenyl)-1H-indol-3-yl](oxo)acetic acid;
{1-Butyl-5-[4-(trifluoromethyl)phenyl]-1H-indol-3-yl}(oxo)acetic acid;
[1-(4-tert-Butylbenzyl)-5-(3-methylphenyl)-1H-indol-3-yl](oxo)acetic acid;
[1-(4-tert-Butylbenzyl)-5-(3-methoxyphenyl)-1H-indol-3-yl](oxo)acetic acid;
[1-(4-tert-Butylbenzyl)-5-(4-tert-butylphenyl)-1H-indol-3-yl](oxo)acetic acid;
[1-(4-tert-Butylbenzyl)-5-(3-chlorophenyl)-1H-indol-3-yl](oxo)acetic acid;
[1-(4-tert-Butylbenzyl)-5-(4-chlorophenyl)-1H-indol-3-yl](oxo)acetic acid;
[1-(4-tert-Butylbenzyl)-5-(2-methylphenyl)-1H-indol-3-yl](oxo)acetic acid;
{1-(2-Ethylbutyl)-5-[4-(trifluoromethoxy)phenyl]-1H-indol-3-yl}(oxo)acetic acid;
{2-[(Acetyloxy)methyl]-1-(4-methylbenzyl)-5-[4-(trifluoromethoxy)phenyl]-1H-indol-3-yl}(oxo)acetic acid;
{2-(Hydroxymethyl)-1-(4-methylbenzyl)-5-[4-(trifluoromethoxy)phenyl]-1H-indol-3-yl}(oxo)acetic acid;
{2-[(Acetyloxy)methyl]-1-benzyl-5-[4-(trifluoromethoxy)phenyl]-1H-indol-3-yl}(oxo)acetic acid;
{1-Benzyl-2-(hydroxymethyl)-5-[4-(trifluoromethoxy)phenyl]-1H-indol-3-yl}(oxo)acetic acid;
[5-(3-Chlorophenyl)-1-cyclopentyl-1H-indol-3-yl]-oxo-acetic acid;
[5-(3-chlorophenyl)-1-(cyclobutylmethyl)-1H-indol-3-yl](oxo)acetic acid;
[5-(3-chlorophenyl)-1-(3-methylcyclopropyl)-1H-indol-3-yl](oxo)acetic acid;
[5-(3-chlorophenyl)-1-(cyclohexylmethyl)-1H-indol-3-yl](oxo)acetic acid;
5-(4-trifluoromethylphenyl)-1-(cyclopentyl)-1H-indol-3-yl](oxo)acetic acid;
[5-(4-trifluoromethylphenyl)-1-(cyclobutylmethyl)-1H-indol-3-yl](oxo)acetic acid;
[5-(4-trifluoromethylphenyl)-1-(3-methylcyclopentyl)-1H-indol-3-yl](oxo)acetic acid;
[5-(4-trifluoromethylphenyl)-1-(cyclohexylmethyl)-1H-indol-3-yl](oxo)acetic acid;
[5-(4-trifluoromethylphenyl)-1-(cyclopentylpropyl)-1H-indol-3-yl](oxo)acetic acid;
[5-(3-trifluoromethylphenyl)-1-(cyclopentyl)-1H-indol-3-yl](oxo)acetic acid;
[5-(3-trifluoromethylphenyl)-1-(cyclobutylmethyl)-1H-indol-3-yl](oxo)acetic acid;
[5-(3-trifluoromethylphenyl)-1-(3-methylcyclopentyl)-1H-indol-3-yl](oxo)acetic acid;
[5-(3-trifluoromethylphenyl)-1-(cyclohexylmethyl)-1H-indol-3-yl](oxo)acetic acid;
[5-(3-trifluoromethylphenyl)-1-(cyclopentylpropyl)-1H-indol-3-yl](oxo)acetic acid; or
[5-(4-methoxyphenyl)-1-(cyclohexylmethyl)-1H-indol-3-yl](oxo)acetic acid;
   or a pharmaceutically acceptable salt, solvate or ester thereof.

The preferred salt forms of the compounds herein include but are not limited to sodium salts, and potassium salts. Other useful salt forms of these compounds include those formed with pharmaceutically acceptable inorganic and organic bases known in the art. Salt forms prepared using inorganic bases include hydroxides, carbonates or bicarbonates of the therapeutically acceptable alkali metals or alkaline earth metals, such as sodium potassium, magnesium, calcium and the like. Acceptable organic bases include amines, such as benzylzmine, mono-, di- and trialkylamines, preferably those having alkyl groups of from 1 to 6 carbon atoms, more preferably 1 to 3 carbon atoms, such as methylamine, dimethylamine, trimethylamine, ethylamine, diethylamine, triethylamine, mono-, di-, and triethanolamine. Also useful are alkylene diamines containing up to 6 carbon atoms, such as hexamethylenediamine; cyclic saturated or unsaturated bases containing up to 6 carbon atoms, including pyrrolidine, peperidine, morpholine, piperazine and their N-alkyl and N-hydroxyalkyl derivatives, such as N-methyl-morpholine and N-(2-hyroxyethyl)-piperidine, or pyridine. Quaternary salts may also be formed, such as tetralkyl forms, such as tetramethyl forms, alkyl-alkanol forms, such as methyl-triethanol or trimethyl-monoethanol forms, and cyclic ammonium salt forms, such as N-methylpyridinium, N-methyl-N-(2-hydroxyethyl)-morpholinium, N,N-di-methylmorpholinium, N-methyl-N-(2-hydroxyethyl)-morpholinium, or N,N-dimethyl-piperidinium salt forms. These salt forms may be prepared using the acidic compound(s) of Formula I and procedures known in the art.

Ester forms of the compounds of this invention include straight chain alkyl esters having from 1 to 6 carbon atoms or branched chain alkyl groups containing 3 or 6 carbon atoms, including methyl, ethyl, propyl, butyl, 2-methylpropyl and 1,1-dimethylethyl esters. Other esters useful with this invention include those of the formula —COOR₇ wherein R₇ is selected from the formulae:

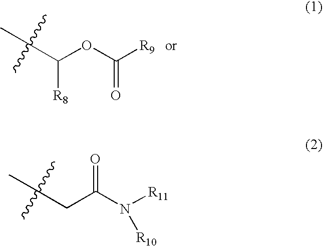

wherein R₈, R₉, R₁₀, R₁₁ are independently selected from hydrogen, alkyl of from 1 to 10 carbon atoms, aryl of 6 to 12 carbon atoms, arylalkyl of from 6 to 12 carbon atoms; heteroaryl or alkylheteroaryl wherein the heteroaryl ring is bound by an alkyl chain of from 1 to 6 carbon atoms.

Among the preferred ester forms of the compounds herein include but not limited to C₁-C₆ alkyl esters, C₃-C₆ branched alkyl esters, benzyl esters, etc.

As used herein, the terms alkyl, alkenyl and alkynyl include both straight chain as well as branched claim chains. Preferably, the C₁-C₃ perfluoroalkyl substituent is —CF₃; the —O—C₁-C₃ perfluoroalkyl substituent is OCF₃; and the —S—C—C₃ perfluoroalkyl substituent is —SCF₃.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

As used herein, “aryl” refers to an unsaturated aromatic carbocyclic group of from 6 to 14 carbon atoms having a single ring (e.g., phenyl) or multiple condensed (fused) rings (e.g., naphthyl or anthryl). Preferred aryl groups include phenyl, naphthyl and the like. As used herein, ‘heteroaryl’, as defined herein, whether alone or as part of another group, refers to a mono- or bicyclic aromatic ring system containing 5-10 ring members of which 1-5 ring members are heteroatoms selected from N, O or S. At least one of the rings of the bicyclic ring system is heteroaromatic. Such heteroaryl groups can have a single ring, such as pyridyl, pyrrolyl or furyl groups, or multiple condensed rings, such as indolyl, indolizinyl, benzofuranyl or benzothienyl groups. Preferred heteroaryls include pyridyl, pyrrolyl and furyl.

Unless otherwise limited by the definition for the aryl or heteroaryl groups herein, such groups can optionally be substituted with from 1 to 5 substituents selected from the group consisting of acyloxy, hydroxy, acyl, alkyl of 1 to 6 carbon atoms, alkoxy of 1 to 6 carbon atoms, alkenyl of 2 to 6 carbon atoms, alkynyl of 2 to 6 carbon atoms, substituted alkyl, substituted alkoxy, substituted alkenyl, substituted alkynyl, amino, amino substituted by one or two alkyl groups of from 1 to 6 carbon atoms, aminoacyl, acylamino, azido, cyano, halo, nitro, thioalkoxy of from 1 to 6 carbon atoms, substituted thioalkoxy of from 1 to 6 carbon atoms, and trihalomethyl. Substituents on the alkyl, alkenyl, alkynyl, thioalkoxy and alkoxy groups mentioned above include halogens, CN, OH, and amino groups. Preferred substituents on the aryl groups herein include alkyl, alkoxy, halo, cyano, nitro, trihalomethyl, and thioalkoxy.

The compounds in this invention may contain one or more asymmetric centers, which can thus give rise to optical isomers (enantiomers) and diastereomers. While shown without respect to the stereochemistry in Formula I, the present invention includes such optical isomers (enantiomers) and diastereomers (geometric isomers); as well as the racemic and resolved, enantiomerically pure R and S stereoisomers; as well as other mixtures of the R and S stereoisomers and pharmaceutically acceptable salts thereof. The use of these compounds is intended to cover the racemic mixture or either of the chiral enantiomers.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties.

The compositions of the present invention are inhibitors of the serine protease inhibitor PAI-1, and are therefore useful in the treatment, inhibition, prevention or prophylaxis in a mammal, preferably in a human, of those processes which involve the production and/or action of PAI-1 (a “PAI-1-associated disorder”). Thus, the compositions of the invention are useful in the treatment or prevention of noninsulin dependent diabetes mellitus and cardiovascular, ocular or kidney disease caused by such condition, and prevention of thrombotic events associated with coronary artery and cerebrovascular disease. These compositions are also useful for inhibiting the disease process involving the thrombotic and prothrombotic states which include, but are not limited to, formation of atherosclerotic plaques, venous and arterial thrombosis, myocardial ischemia, atrial fibrillation, deep vein thrombosis, coagulation syndromes, pulmonary fibrosis, cerebral thrombosis, thromboembolic complications of surgery (such as joint replacement), and peripheral arterial occlusion. These compositions are also useful in treating ischemic events such as stroke, associated with or resulting from atrial fibrillation.

The compositions of the invention may also be used in the treatment of diseases associated with extracellular matrix accumulation, including, but not limited to, renal fibrosis, chronic obstructive pulmonary disease, polycystic ovary syndrome, restenosis, renovascular disease and organ transplant rejection.

The compositions of the invention may also be used in the treatment of malignancies, and diseases associated with neoangiogenesis (such as diabetic retinopathy and age-related macular degeneration).

The compositions in the invention may also be used in conjunction with and following processes or procedures involving maintaining blood vessel patency, including vascular surgery, vascular graft and stent patency, organ, tissue and cell implantation and transplantation.

The compositions in the invention may also be useful in the treatment of inflammatory diseases, septic shock and the vascular damage associated with infections.

The compositions of the invention are useful for the treatment of blood and blood products used in dialysis, blood storage in the fluid phase, especially ex vivo platelet aggregation. The present compositions may also be added to human plasma during the analysis of blood chemistry in hospital settings to determine the fibrinolytic capacity thereof.

The compositions in the present invention may also be used in combination with prothrombolytic, fibrinolytic and anticoagulant agents.

The compositions of the present invention may also be used to treat cancer including, but not limited to, breast and ovarian cancer, and as imaging agents for the identification of metastatic cancers.

The compositions of the invention may also be used in the treatment of Alzheimer's disease. This method may also be characterized as the inhibition of plasminogen activator by PAI-1 in a mammal, particularly a human, experiencing or subject to Alzheimer's disease. This method may also be characterized as a method of increasing or normalizing levels of plasmin concentration in a mammal, particularly those experiencing or subject to Alzheimer's disease.

The compositions of the invention may be used for reducing amyloid beta levels in a mammal, preferably a human, suffering from Alzheimer's disease, comprising the administration of a therapeutically effective amount of the composition. In some embodiments, the methods of this invention reduce amyloid beta levels in the brain.

The compositions of the invention may be used for improving cognition in a mammal, preferably a human, comprising the administration of a therapeutically effective amount of the composition.

The compositions of the invention may be used for treating pre-senile or senile dementia in a mammal, preferably a human.

The compositions of the invention are useful as medicament, and also in the manufacture of a medicament useful for the treatment of Alzheimer's disease in a mammal, preferably a human.

The compositions of the invention may be used for the treatment of myelofibrosis with myeloid metaplasia by regulating stromal cell hyperplasia and increases in extracellular matrix proteins.

The compositions of the invention may also be used in conjunction with protease inhibitor—containing highly active antiretroviral therapy (HAART) for the treatment of diseases which originate from fibrinolytic impairment and hyper-coagulability of HIV-1 infected patients receiving such therapy.

The compositions of the invention may be used for the treatment of diabetic nephropathy and renal dialysis associated with nephropathy.

The compositions of the invention may be used to treat cancer, septicemia, obesity, insulin resistance, proliferative diseases such as psoriasis, improve coagulation homeostasis, treat cerebrovascular diseases, microvascular disease, hypertension, dementia, osteoporosis, arthritis, asthma, heart failure, arrhythmia, angina, as a hormone replacement agent, and for treating, preventing or reversing progression of atherosclerosis, Alzheimer's disease, osteoporosis, and osteopenia; reduce inflammatory markers, reducing C-reactive protein, or for preventing or treating low grade vascular inflammation, stroke, dementia, coronary heart disease, for primary and secondary prevention of myocardial infarction, stable and unstable angina, primary prevention of coronary events, secondary prevention of cardiovascular events, peripheral vascular disease, peripheral arterial disease, acute vascular syndromes, reduce the risk of undergoing a myocardial revascularization procedure, treat microvascular diseases such as nephropathy, neuropathy, retinopathy and nephrotic syndrome, hypertension, Type 1 and 2 diabetes and related diseases, hyperglycemia, hyperinsulinemia, malignant lesions, premalignant lesions, gastrointestinal malignancies, liposarcomas and epithelial tumors, proliferative diseases such as psoriasis, improve coagulation homeostasis, and/or endothelial function, and treat all forms of cerebrovascular diseases.

The compositions of the invention include those that are useful in the manufacture of a medicament and in some embodiments are medicaments themselves.

This invention provides novel formulations containing indole-based PAI-1 inhibitors of formula I. One of skill in the art can appreciate the difficulties inherent in providing formulations for compounds that are lipophilic and acidic. Such compounds, due to the presence of a polar section(s) together with a hydrophobic portion can present numerous difficulties to the task of providing a formulation that is capable of providing significant levels of the active moiety into the subject's bloodstream. One of the difficulties is in providing a formulation that will protect the compound from decomposition while simultaneously helping to solubilize the drug for purposes of enhancing absorption. Clearly, the need to solubilize the drug must be weighed against not introducing excess excipients which might exacerbate loading and stability problems. The present invention describes highly useful, novel and effective formulations for the delivery of compounds of formula (I). In particular, the present invention provides liquid or emulsified dosage formulations especially suitable to the dosing of mammals of a compound of formula (I). In certain embodiments of the invention, the mammal to be dosed is a human.

As used herein, “A composition suitable for use as an oral formulation” means (1) “A composition suitable for oral administration”, (2) “A composition suitable for use in an oral formulation” or (3) “A composition suitable for use as/in an oral formulation.”

In one embodiment, compositions of this invention comprise a compound of formula (I) in a range of about 0.01% to 30% w/w of the composition. In another embodiment, the composition of this invention comprises a compound of formula (I) in a range of about 0.01% to 20% w/w of the composition. In yet another embodiment of this invention, the composition comprises a compound of formula (I) in a range of about 0.01% to 10% w/w of the composition. In some embodiments, the composition of this invention comprises a compound of formula (I) in about 0.01% w/w of the composition. In some embodiments, the composition of this invention comprises a compound of formula (I) in about 6% w/w of the composition. In some embodiments, the composition of this invention comprises a compound of formula (I) in about 10% w/w of the composition. In some embodiments, the composition of this invention comprises a compound of formula (I) in about 20% w/w of the composition

Combined within the compositions of this invention are the compound embodiments of the invention with one or more solubilizers/emulsifiers. Since this invention contemplates the inclusion of multiple solubilizers/emulsifiers, the solubilizer/emulsifier discussed in this paragraph will be referred to as solubilizer/emulsifier A. In certain embodiments, the compositions of this invention are in the form of an emulsion comprising one or more compounds of the invention together with one or more excipients useful in the preparation and presentation of the formulations of this invention. The oral formulations of the instant invention will contain one or more solubilizers or emulsifiers. In some embodiments, the solubilizer/emulsifier A is a non-ionic surfactant. In some embodiments, the solubilizer/emulsifier A is a glycerol-polyethylene glycol ester of a fatty acid. In further embodiments, the fatty acid maybe a hydroxylated fatty acid. For example, a glycerol-polyethylene glycol ricinoleate is a useful component for solubilization of a compound of the composition of this invention, wherein said glycerol-polyethylene glycol ricinoleate may be present together with fatty acid esters of polyethyleneglycol as well as polyethylene glycols and ethoxylated glycerol. An example of a very useful solubilizer comporting with this definition is Cremophor® EL. If desired, a flavor masking agent maybe used in the context of this invention or alternative, hydrogenated Cremophors such as Cremophor® RH40 might be used in lieu of a non-hydrogenated product. In some embodiments, the solubilizer/emulsifier A is present in from about 25% to 50% w/w of the composition. In certain embodiments, the solubilizer/emulsifier A is present in from about 30% to 45% w/w of the composition. In yet other embodiments, the solubilizer/emulsifier A is present in from about 36% to 40% w/w of the composition. In yet other embodiments, the solubilizer/emulsifier A is present in from about 40% w/w of the composition. In yet other embodiments, the solubilizer/emulsifier A is present in from about 36% w/w of the composition. In yet other embodiments, the solubilizer/emulsifier A is present in from about 32% w/w of the composition.

As was mentioned previously, this invention contemplates the use of more than one solubilizing/emulsifying agent. Thus, in addition to the emulsifying/solubilizing agents just discussed, a solubilizer/emulsifier B can be effectively employed interchangeably or in addition to the solubilizer/emulsifier A for use in this invention. In some embodiments, the solubilizer/emulsifier B comprises a non-ionic surfactant. In certain embodiments, the solubilizer/emulsifier B comprises an ester of a hydroxylated fatty acid optionally with a polyalkylene glycol. In certain embodiments, the acid maybe a 12- or 15-hydroxy steareate. In certain embodiments, the solubilizer/emulsifier B consists of polyglycol mono- and di-esters of 12-hydroxystearic acid wherein said polyglycol mono- and di-esters of 12-hydroxystearic acid can further comprise from about 20 to 40% or about 30% free polyethylene glycol. In some embodiments, the solubilizer/emulsifier B is Solutol® HS15 or macrogol 15 hydroxystearate. In some embodiments, the solubilizer/emulsifier B is present in from about 25% to 50% w/w of the composition. In certain embodiments, the solubilizer/emulsifier B is present in from about 30% to 45% w/w of the composition. In yet other embodiments, the solubilizer/emulsifier B is present in from about 36% to 40% w/w of the composition. In yet other embodiments, the solubilizer/emulsifier B is present in about 40% w/w of the composition. In yet other embodiments, the solubilizer/emulsifier B is present in about 36% w/w of the composition. In yet other embodiments, the solubilizer/emulsifier B is present in about 32% w/w of the composition

In another embodiment of this invention, another emulsifier/solubilizer, C, maybe used either interchangeably with A, B or A and B, or in addition to A and B. In some embodiments, the emulsifier/solubilizer C comprises a non-ionic surfactant. In further embodiments, the emulsifier/solubilizer C comprises a polysorbate. In certain embodiments, the polysorbate is a polysorbate 20, 21, 40, 60, 61, 65, 80, 81, 85 or 120. In further embodiments, the polysorbate is a polysorbate 60, 61, 65, 80, 81 or 85. In yet further embodiments, the polysorbate is polysorbate 80. In some embodiments, the polysorbate is present in from 10% to 90% w/w of the composition. In still other embodiments, the polysorbate is present in from about 20% to 80% w/w of the composition. In still yet other embodiments, the polysorbate is present in from about 30% to 70% w/w of the composition. In certain embodiments, the polysorbate is present in from about 30% to 50% w/w of the composition. In some embodiments, the polysorbate is present in from about 36% to 40% w/w of the composition. In other embodiments, the polysorbate is present in about 2% w/w of the composition. In other embodiments, the polysorbate is present in about 36% w/w of the composition. In yet further embodiments, the polysorbate is present in about 40% w/w of the composition.

This invention can also include another solubilizer/emulsifier, D. In some embodiments, the solubilizer/emulsifier D is selected from an alkylene glycol ester. In certain embodiments, the solubilizer/emulsifier D is a propylene glycol mono- or di-ester. In yet other embodiments, the solubilizer/emulsifier D is a propylene glycol mono-ester. In certain embodiments, the solubilizer/emulsifier D is selected from propylene glycol dioleate, 2-hydroxypropyl stearate, 2-hydroxypropyl laurate, propylene glycol monostearate, propylene glycol oleate, propylene glycol distearate, propylene glycol dicaprylate, propylene glycol monolaurate, propylene glycol dilaurate, polypropylene glycol (17) dioleate, propyleneglycol monolaurate, Propylene glycol monomyristate, dipropylene glycol dipelargonate, propylene glycol monocaprylate, polypropylene glycol monobutyl ether oleate, propylene glycol dipelargonate, propylene glycol didecanoate, dipropylene glycol dipelargonate, propylene glycol bis(9,10-epoxystearate), propylene glycol monoisostearate and propylene glycol diundecanoate. In certain embodiments, solubilizer/emulsifier D is propylene glycol monocaprylate (Capryol® 90). In certain embodiments of this invention, the solubilizer/emulsifier D is present in an amount from about 5% to 35% w/w of the composition. In other embodiments, the solubilizer/emulsifier D is present in an amount from about 10% to 28% w/w of the composition. In still yet other embodiments, the solubilizer/emulsifier D is present in an amount from 10% to 28% w/w of the composition. In other embodiments, the solubilizer/emulsifier D is present in an amount from about 15% to 25% w/w of the composition. In yet other embodiments, the solubilizer/emulsifier D is present in an amount from about 18% to 20% w/w of the composition. In some embodiments, the solubilizer/emulsifier D is present in an amount from about 16% w/w of the composition.

In still yet other embodiments, certain other agents can be used with, or substituted for (alone or in combination), the solubilizer D. In this regard, lauroglycol 90, Capmul MCM, Capmul PG-8, Captex 355 and labrasol all can be useful either alone or in combination, with or without D, as solubilizers for the formulations of this invention.

The various structural embodiments of this invention as described by the various formulae presented, may be formulated according to the procedures described in this application.

### EXAMPLES

The following examples are to be considered non-limiting. For purposes of this invention, embodiments maybe combined to achieve additional embodiments. The compositions of this invention, at a minimum, comprise a compound of the invention and one or more emulsifier or solubilizer. Representative formulations of the invention are listed below.

### Example 1

| Ingredients | Amount w/w |
|---|---|
| Compound Formula I | 0.01% to 30% |
| A | 25% to 50% |
| B | 25% to 50% |
| Additional Excipients | Remainder |

### Example 2

| Ingredients | Amount w/w |
|---|---|
| Compound Formula I | 0.01% to 30% |
| A | 25% to 50% |
| C | 25% to 50% |
| Additional Excipients | Remainder |

### Example 3

| Ingredients | Amount w/w |
|---|---|
| Compound Formula I | 0.01% to 30% |
| A | 25% to 50% |
| D | 25% to 50% |
| Additional Excipients | Remainder |

### Example 4

| Ingredients | Amount w/w |
|---|---|
| Compound Formula I | 0.01% to 30% |
| A | 25% to 50% |
| B | 25% to 50% |
| D | 5% to 35% |
| Additional Excipients | Remainder |

### Example 5

| Ingredients | Amount w/w |
|---|---|
| Compound Formula I | 0.01% to 30% |
| A | 25% to 50% |
| C | 25% to 50% |
| D | 5% to 35% |
| Additional Excipients | Remainder |

### Example 6

| Ingredients | Amount w/w |
|---|---|
| Compound Formula I | 0.01% to 30% |
| C | 25% to 50% |
| B | 25% to 50% |
| D | 5% to 35% |
| Additional Excipients | Remainder |

### Example 7

| Ingredients | Amount w/w |
|---|---|
| Compound Formula I | 0.01% to 20% |
| A | 30% to 45% |
| B | 30% to 45% |
| D | 10% to 28% |
| Additional Excipients | Remainder |

### Example 8

| Ingredients | Amount w/w |
|---|---|
| Compound Formula I | 0.01% to 20% |
| A | 30% to 45% |
| C | 30% to 45% |
| D | 10% to 28% |
| Additional Excipients | Remainder |

### Example 9

| Ingredients | Amount w/w |
|---|---|
| Compound Formula I | 0.01% to 20% |
| C | 30% to 45% |
| B | 30% to 45% |
| D | 10% to 28% |
| Additional Excipients | Remainder |

### Example 10

| Ingredients | Amount w/w |
|---|---|
| Compound Formula I | 0.01% to 10% |
| A | 36% to 40% |
| B | 36% to 40% |
| D | 15% to 25% |
| Additional Excipients | Remainder |

### Example 11

| Ingredients | Amount w/w |
|---|---|
| Compound Formula I | 0.01% to 10% |
| A | 36% to 40% |
| B | 36% to 40% |
| D | 18% to 20% |
| Additional Excipients | Remainder |

### Example 12

| Ingredients | Amount w/w |
|---|---|
| [1-(4-tert-Butylbenzyl)-5-(3-methylphenyl)-1H- | 0.01% to 30% |
| indol-3-yl](oxo)acetic acid | |
| Cremophor ® EL | 25% to 50% |
| Solutol ® HS-15 | 25% to 50% |
| Capryol ® 90 | 5% to 35% |
| Additional Excipients | Remainder |

### Example 13

| Ingredients | Amount w/w |
|---|---|
| [1-(4-tert-Butylbenzyl)-5-(3-methylphenyl)-1H- | 0.01% to 30% |
| indol-3-yl](oxo)acetic acid | |
| Tween 80 | 25% to 50% |
| Solutol ® HS-15 | 25% to 50% |
| Capryol ® 90 | 5% to 35% |
| Additional Excipients | Remainder |

### Example 14

| Ingredients | Amount w/w |
|---|---|
| [1-(4-tert-Butylbenzyl)-5-(3-methylphenyl)-1H- | 0.01% to 30% |
| indol-3-yl](oxo)acetic acid | |
| Cremophor ® EL | 25% to 50% |
| Tween ® 80 | 25% to 50% |
| Capryol ® 90 | 5% to 35% |
| Additional Excipients | Remainder |

### Example 15

| Ingredients | Amount w/w |
|---|---|
| [1-(4-tert-Butylbenzyl)-5-(3-methylphenyl)-1H- | 0.01% to 20% |
| indol-3-yl](oxo)acetic acid | |
| Cremophor ® EL | 30% to 45% |
| Solutol ® HS-15 | 30% to 45% |
| Capryol ®90 | 10% to 28% |
| Additional Excipients | Remainder |

### Example 16

| Ingredients | Amount w/w |
|---|---|
| [1-(4-tert-Butylbenzyl)-5-(3-methylphenyl)-1H- | 0.01% to 10% |
| indol-3-yl](oxo)acetic acid | |
| Cremophor ® EL | 36% to 40% |
| Solutol ® HS-15 | 36% to 40% |
| Capryol ® 90 | 18% to 20% |
| Additional Excipients | Remainder |

### Example 17

| Ingredients | Amount w/w |
|---|---|
| [1-(4-tert-Butylbenzyl)-5-(3-methylphenyl)-1H- | 0.01% |
| indol-3-yl](oxo)acetic acid | |
| Cremophor ® EL | 40% |
| Solutol ® HS-15 | 40% |
| Capryol ® 90 | 20% |

### Example 18

| Ingredients | Amount w/w |
|---|---|
| [1-(4-tert-Butylbenzyl)-5-(3-methylphenyl)-1H- | 10% |
| indol-3-yl](oxo)acetic acid | |
| Cremophor ® EL | 36% |
| Solutol ® HS-15 | 36% |
| Capryol ® 90 | 18% |

### Example 19

| Ingredients | Amount w/w |
|---|---|
| [1-(4-tert-Butylbenzyl)-5-(3-methylphenyl)-1H- | 20% |
| indol-3-yl](oxo)acetic acid | |
| Cremophor ® EL | 32% |
| Solutol ® HS-15 | 32% |
| Capryol ® 90 | 16% |

### Preparation of Formulations

The compounds useful in the compositions of this invention can be prepared according to procedures known to those of ordinary skill in the art, and in particular to those described in U.S. application No. 2003/0125371, now U.S. Pat. No. 7,074,817, each of which is herein incorporated by reference in its entirety.

The following examples are not to be construed as limiting the invention to any particular process of preparation nor any particular oral dosage form. Ingredients for use in the formulations of this invention may be dry blended or wet blended. Individual or groups of components may be first dry blended and then wet blended together, thus the processes for the preparation of the formulations of this invention are contemplated to include mixed blending regimens. The formulations of this invention may also be prepared by, for example, a melt granulation where two or more ingredients are combined and then melted together and then further processed. The preparation of representative formulations of the invention are shown below. However, the formulations of this invention are not to be construed or limited by the processes specifically delineated herein but rather include any and all processes ascertainable by one of ordinary skill in the art.

### Example 20

### Preparation of a Liquid Composition Suitable for Oral Delivery Containing a Compound of Formula I

1. A solubilizer/emulsifier B if it begins as a solid, is melted and mixed thoroughly prior to use, making sure that the entire contents are melted in order to ensure homogeneity of the ingredient.
2. The melted/liquid, solubilizer/emulsifier B is combined with a solubilizer/emulsifier A and a solubilizer/emulsifier D in an appropriate mixing vessel.
3. A, B and D are mixed together until a homogeneous solution is obtained.
4. A compound of formula I is weighed out and slowly added to the vessel while mixing.
5. The mixing is continued until the compound of formula I is dissolved.
6. The composition is stored until needed, preferably protected from light.

### Preparation of a Liquid Composition Suitable for Oral Delivery Containing [1-(4-tert-Butylbenzyl)-5-(3-methylphenyl)-1H-indol-3-yl](oxo)acetic acid

1. Solutol® HS-15 was melted at 50° C. and mixed thoroughly before use, ensuring that the entire content of the container holding the Solutol® HS-15 was mixed since Solutol® HS-15 was not homogeneous in the solid-state.
2. Cremophor® EL, melted Solutol® HS 15, and Capryol® 90 were added in the desired quantities to an appropriate mixing vessel.
3. Cremophor® EL, melted Solutol® HS 15, and Capryol® 90 were mixed together until a homogeneous solution was obtained.

4. [1-(4-tert-Butylbenzyl)-5-(3-methylphenyl)-1H-indol-3-yl](oxo)acetic acid was weighed out and slowly added to the vessel while mixing.
5. The mixing was continued until [1-(4-tert-Butylbenzyl)-5-(3-methylphenyl)-1H-indol-3-yl](oxo)acetic acid was dissolved.
6. The composition was stored until needed, protected from light.

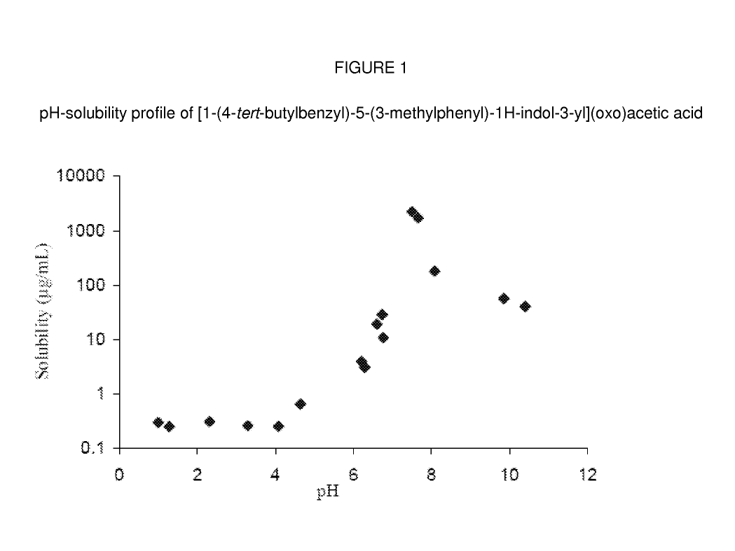

### Solubility Profile

[1-(4-tert-Butylbenzyl)-5-(3-methylphenyl)-1H-indol-3-yl](oxo)acetic acid is an α-oxo carboxylic acid with a calculated pKa of 3.53 for the indole oxoacetic acid, and an aqueous solubility of approximately 0.25 uG/mL in the ionized form which increases to approximately 24 uG/mL upon ionization in aqueous media. A solubility profile for [1-(4-tert-butylbenzyl)-5-(3-methylphenyl)-1H-indol-3-yl](oxo)acetic acid was generated using the free acid in HCl/NaOH solution (See FIG. 1.) Solutions were centrifuged after 24 hours equilibration (2 hours for pH<4 to avoid degradation) and the supernatants were assayed by HPLC. Above pH 4, solubility increases with increasing pH up to pH˜8. Above pH 8, solubility decreases due to precipitation of the sodium salt, which is of very small particle size. Throughout the pH range, samples remained “cloudy” after filtration through a 0.2 μfilter, hence centrifugation for 2 hours was used as a means of phase separation. The low solubility at acidic pH's indicates that dissolution of [1-(4-tert-Butylbenzyl)-5-(3-methylphenyl)-1H-indol-3-yl](oxo)acetic acid will not occur appreciably in the stomach, but rather in the small intestine as the pH reaches near-neutral values. The solubility in water is still low at 24 μg/mL at neutral pH 6.9. However, solubility is greatly enhanced (26.000-fold) in the 2% Tween80/0.5% methylcellulose solution to 0.58 mg/mL (pH 3.5). In some embodiments, the [1-(4-tert-Butylbenzyl)-5-(3-methylphenyl)-1H-indol-3-yl](oxo)acetic acid used with the composition of the invention is micronized.

### Bioavailability

Three formulations were evaluated for the bioavilability of [1-(4-tert-butylbenzyl)-5-(3-methylphenyl)-1h-indol-3-yl](oxo)acetic acid in male dogs. Following a single oral (gavage, 300 mg/kg) dose of [1-(4-tert-butylbenzyl)-5-(3-methylphenyl)-1h-indol-3-yl](oxo)acetic acid, blood samples were drawn at 0 (predose), 0.25, 0.5, 1, 2, 3, 4, 6, 8, 10, 12, 24 and 30 hours. Pharmacokinetic parameters with the corresponding formulation were shown in Table 1. The pharmacokinetic parameters, AUC, Cₘₐₓ, tₘₐₓ and t_{1/2}, were determined for each dog, and descriptive statistics were calculated for comparison among formulations. Cremophor Based formulation (Formula C) resulted the highest AUC and lowest deviation.

TABLE 1
Individual and Mean (±SD) [1-(4-tert-Butylbenzyl)-5-(3-methylphenyl)-1H-indol-3-yl](oxo)acetic
acid Pharmacokinetic Parameters in Fasted Male Dogs

| | | Cₘₐₓ | tₘₐₓ | AUC₀₋₃₀ | AUC_{0-∞} | t_{1/2} |
|---|---|---|---|---|---|---|
| FORMULATION | SAN | (μg/mL) | (hr) | (μg · hr/mL) | (μg · hr/mL) | (hr) |
| Tween/NaHCO₃^{a} | 1 | 38.9 | 4.0 | 373 | 379 | 4.7 |
| (Formula A) | 2 | 59.9 | 6.0 | 617 | 631 | 5.0 |
| | 3 | 21.6 | 4.0 | 216 | 222 | 5.9 |
| | 9 | 56.4 | 10.0 | 903 | 973 | 6.7 |
| | Mean ± SD | 44.2 ± 17.6 | 6.0 ± 2.8 | 527 ± 300 | 551 ± 328 | 5.6 ± 0.9 |
| Phosal Based^{b} | 5 | 24.5 | 4.0 | 247 | 249 | 4.1 |
| (Formula B) | 6 | 21.4 | 2.0 | 203 | 213 | 7.0 |
| | 7 | 33.8 | 6.0 | 412 | ND | ND |
| | 8 | 91.6 | 12.0 | 1559 | ND (~1654) | ND |
| | Mean ± SD | 42.8 ± 32.9 | 6.0 ± 4.3 | 605 ± 642 | 231 (n = 2) | 5.6 |
| | | | | | | (n = 2) |
| Cremophor Based^{c} | 10 | 43.6 | 12.0 | 886 | ND (~1155) | ND |
| (Formula C) | 11 | 34.7 | 2.0 | 389 | 399 | 5.4 |
| | 12 | 48.0 | 6.0 | 776 | ND (~1021) | ND |
| | 13 | 43.0 | 4.0 | 529 | 551 | 6.0 |
| | Mean ± SD | 42.3 ± 5.6 | 6.0 ± 4.3 | 645 ± 227 | 475 (n = 2) | 5.7 |
| | | | | | | (n = 2) |

Formulations Used for the above study

Percentage

Tween/NaHCO₃^{a}
(Formula A)

| | |
|---|---|
| [1-(4-tert-Butylbenzyl)-5-(3-methylphenyl)-1H- | 6% |
| indol-3-yl](oxo)acetic acid (use at 99.5%) | |
| Polysorbate 80 | 2% |
| Methylcellulose (4000 cps) | 0.50% |
| Sodium Bicarbonate | 0.42% (50 mM) |
| Water | Qs to 100% |

Phosal Based^{b}
(Formula B)

| | |
|---|---|
| [1-(4-tert-Butylbenzyl)-5-(3-methylphenyl)-1H- | 20% |
| indol-3-yl](oxo)acetic acid (use at 99.5%) | |
| Phosal 53 MCT | 47% |
| Labrasol | 14% |
| Propylene Carbonate | 14% |
| Polysorbate 80 | 5% |

Cremophor Based^{c}
(Formula C)

| | |
|---|---|
| [1-(4-tert-Butylbenzyl)-5-(3-methylphenyl)-1H- | 20% |
| indol-3-yl](oxo)acetic acid (use at 99.5%) | |
| Cremophor EL | 32% |
| Solutol HS-15 | 32% |
| Capryol 90 | 16% |

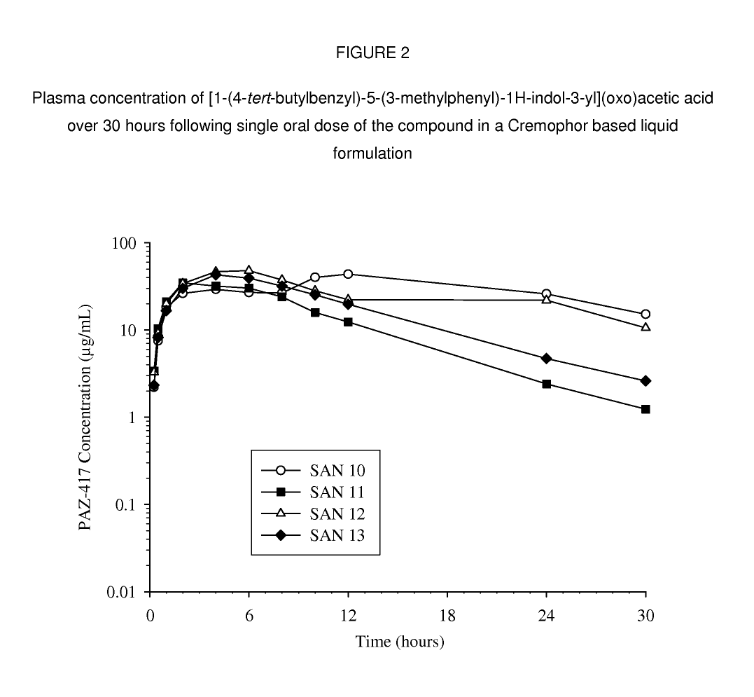

Individual plasma concentrations over time after dosing Formula C are tabulated and plotted in Table 2 and FIG. 2 respectively.

TABLE 2
Plasma [1-(4-tert-Butylbenzyl)-5-(3-methylphenyl)-1H-indol-3-yl](oxo)acetic
acid concentrations (μg/mL) in Fasted Male Dogs

| | Hours After Dose | | | | | | | | | | |
|---|---|---|---|---|---|---|---|---|---|---|---|
| Dog | 0.25 | 0.5 | 1 | 2 | 4 | 6 | 8 | 10 | 12 | 24 | 30 |
| 10 | 2.20 | 7.50 | 18.1 | 26.3 | 29.3 | 26.9 | 26.5 | 40.1 | 43.6 | 25.9 | 15.2 |
| 11 | 3.39 | 10.4 | 21.1 | 34.7 | 31.9 | 30.3 | 24.0 | 15.9 | 12.4 | 2.41 | 1.24 |
| 12 | 3.29 | 9.19 | 20.0 | 34.1 | 46.6 | 48.0 | 37.6 | 28.2 | 22.3 | 21.9 | 10.6 |
| 13 | 2.33 | 8.19 | 16.7 | 30.0 | 43.0 | 39.4 | 31.9 | 25.3 | 19.7 | 4.72 | 2.61 |
| Mean | 2.80 | 8.82 | 19.0 | 31.3 | 37.7 | 36.2 | 30.0 | 27.4 | 24.5 | 13.7 | 7.41 |
| SD | 0.624 | 1.26 | 1.96 | 3.92 | 8.40 | 9.50 | 6.04 | 9.98 | 13.4 | 11.9 | 6.63 |
| n | 4 | 4 | 4 | 4 | 4 | 4 | 4 | 4 | 4 | 4 | 4 |

The compositions of this invention may be dosed in mammals according to protocols known to those of ordinary skill in the art. For example, the emulsions/liquids of this invention may be prepared and delivered as capsules, gels, syrups, gums, suppositories and the like. In addition to the composition agents as described herein, the compositions of this invention may also be combined with one or more flavor masking agents including sweeteners, such as, for example sucrose, saccharin and the like as well as scents or aromas including peppermint oil, contramarum aroma, cinnamon aroma, boonekamp aroma, orange aroma or lemon aroma and the like. The compositions of this invention may also include one or more preservatives, such as water soluble or oil soluble antioxidants, or combinations thereof. For example, antioxidants suitable for contemplated use in this invention include BHT, ascorbic acid, vitamin E and the like. The compositions of this invention may also contain pH adjusters, acidic or basic that can serve to fix the pH of the compositions of the invention. Such pH adjusters may comprise inorganic salts as well as organic acids or salts of organic acids. Additionally, the pH adjusters maybe present in the form of a buffer. The compositions of this invention may also comprise a complexing agent, such as, for example, EDTA and the like wherein such complexing agents might serve to further solubilize the compound and minimize precipitation of one or more substances from the composition. The compositions of this invention may also contain viscosity agents wherein such agents can serve to help adjust the viscosity to the desired level. The compositions of this invention may also include coloring agents including pharmaceutically acceptable, synthetic or naturally occurring dyes and the like.

Methods for the treatment, inhibition, prevention or prophylaxis in a mammal of each of the conditions or maladies listed herein are part of the present invention. Each method comprises administering to a mammal in need thereof a pharmaceutically or therapeutically effective amount of a compound of this invention, or a pharmaceutically acceptable salt or ester form thereof. Where a method of treatment is referred to herein, that method will also cover the prevention or prophylaxis of the same disorder, disease or condition being treated.

Each of the methods described herein comprise administering to a mammal in need of such treatment a pharmaceutically effective amount of a compound of this invention, or a pharmaceutically acceptable salt or ester form thereof. It will be understood that a pharmaceutically effective amount of the compound will be at least the minimum amount necessary to provide an improvement in the symptoms or underlying causation of the malady in question or to inhibit or lessen the onset of symptoms of the malady.

Dosage amounts vary in accord to the compound used, the age of the patient, the type of illness being treated, the age and condition of the patient and so forth. As a general matter, dose ranges of 1.0 mg to 500 mg may be contemplated. In some embodiments, the dose ranges contemplated may be between 2.5 mg and 200 mg.

It should be appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

All references referred to herein, including patents, patent applications books and other printed publications are incorporated by reference in their entirety.

The application claim priority benefit of U.S. provisional application 60/899,491, incorporated by reference herein in its entirety.

What is claimed:

1. A composition comprising a compound of formula (I), or a pharmaceutically acceptable salt, solvate or ester thereof: 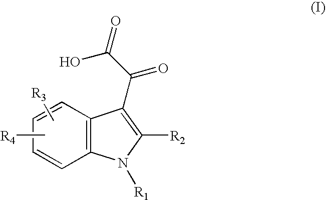
wherein:
R₁ is selected from C₁-C₈ alkyl, (—CH₂)ₙ—C₃-C₆ cycloalkyl, wherein n is an integer of from 0 to 3, pyridinyl, —CH₂-pyridinyl, phenyl or benzyl, the rings of the cycloalkyl, pyridinyl, phenyl and benzyl groups being optionally substituted by, from 1 to 3 groups independently selected from, halogen, C₁-C₄ alkyl, C₁-C₃ perfluoroalkyl, —O—C₁-C₃ perfluoroalkyl, C₁-C₃ alkoxy, —OH, —NH₂, and —NO₂;
R₂ is selected from H, C₁-C₆ alkyl, C₃-C₆ cycloalkyl, —CH₂—C₃-C₆ cycloalkyl, C₁-C₃ perfluoroalkyl, —CH₂OH and CH₂OAc;
R₃ is selected from H, halogen, C₁-C₆ alkyl, C₁-C₃ perfluoroalkyl, C₁-C₆ alkoxy, C₃-C₆ cycloalkyl, —CH₂—C₃-C₆ cycloalkyl, C₄-C₆ cycloalkenyl, —CH₂—C₄-C₆ cycloalkenyl, —NH₂, and —NO₂;
R₄ is phenyl, substituted by from 1 to 3 groups independently selected from halogen, C₁-C₄ alkyl, C₁-C₃ perfluoroalkyl, —CF₃, and —O—C₁-C₃ perfluoroalkyl; and
at least one solubilizer or emulsifier;
wherein said composition comprises a liquid or emulsion.

2. The composition of claim 1 comprises 2, 3, 4 or more solubilizers or emulsifiers.

3. The composition of claim 1, wherein the solubilizers or emulsifiers are selected from A, B, C and D; wherein
A is selected from a non-ionic surfactant, a glycerol-polyethylene glycol ester of a fatty acid wherein the fatty acid is a hydroxylated fatty acid, a glycerol-polyethylene glycol ricinoleate wherein said glycerol-polyethylene glycol ricinoleate is present together with fatty acid esters of polyethyleneglycol as well as polyethylene glycols and ethoxylated glycerol, or polyethoxylated castor oil;
B is selected from a non-ionic surfactant, an ester of a hydroxylated fatty acid optionally with a polyalkylene glycol wherein the acid is a 12- or 15-hydroxy steareate, a polyglycol mono- and di-esters of 12-hydroxystearic acid wherein said polyglycol mono- and di-esters of 12-hydroxystearic acid can further comprise from about 20 to 40% or about 30% free polyethylene glycol, or polyethylene glycol-15-hydroxystearate (macrogol 15 hydroxystearate);
C is selected from a non-ionic surfactant, a polysorbate, polysorbate 20, 21, 40, 60, 61, 65, 80, 81, 85 or 120; and
D is selected from an alkylene glycol ester, a propylene glycol mono- or di-ester, a propylene glycol mono-ester, propylene glycol dioleate, 2-hydroxypropyl stearate, 2-hydroxypropyl laurate, propylene glycol monostearate, propylene glycol oleate, propylene glycol distearate, propylene glycol dicaprylate, propylene glycol monolaurate, propylene glycol dilaurate, polypropylene glycol (17) dioleate, propyleneglycol monolaurate, propylene glycol monomyristate, dipropylene glycol dipelargonate, propylene glycol monocaprylate, polypropylene glycol monobutyl ether oleate, propylene glycol dipelargonate, propylene glycol didecanoate, dipropylene glycol dipelargonate, propylene glycol bis(9,10-epoxystearate), propylene glycol monoisostearate, propylene glycol diundecanoate, propylene glycol monocaprylate, Capryol 90, Phosal 53 MCT, Lauroglycol, Capmul MCM, Capmul PG-8, Captex 355, Labrasol or Propylene Carbonate.

4. The composition of claim 1, wherein the compound of formula (I) is a compound of formula (II) or (III), or a pharmaceutically acceptable salt, solvate or ester thereof:

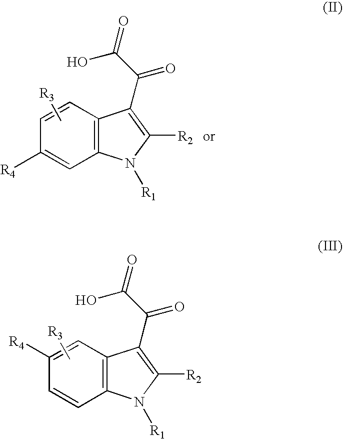

5. The composition of claim 1, wherein the compound of formula (I) is a compound of formula compound of formula (IV) or formula (V), or a pharmaceutically acceptable salt, solvate or ester thereof: 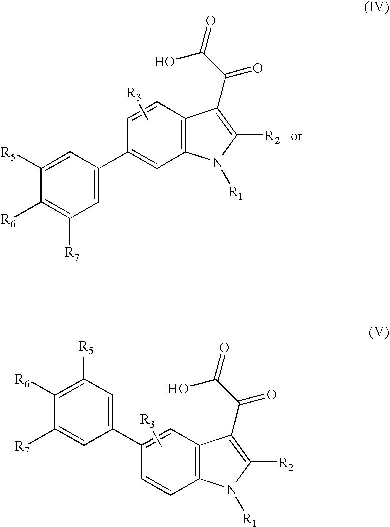
wherein:
R₁ is selected from C₁-C₈ alkyl, C₃-C₆ cycloalkyl, —CH₂—C₃-C₆ cycloalkyl, or benzyl, the rings of the cycloalkyl and benzyl groups being optionally substituted by from 1 to 3 groups selected from halogen, C₁-C₄ alkyl, C₁-C₃ perfluoroalkyl, —O—C₁-C₃ perfluoroalkyl, C₁-C₃ alkoxy, —OH, —NH₂, or —NO₂;
R₂ is selected from H, C₁-C₆ alkyl, C₃-C₆ cycloalkyl, —CH₂—C₃-C₆ cycloalkyl, or C₁-C₃ perfluoroalkyl;
R₃ is selected from H, halogen, C₁-C₆ alkyl, C₁-C₃ perfluoroalkyl, C₁-C₆ alkoxy, C₃-C₆ cycloalkyl, —CH₂—C₃-C₆ cycloalkyl, —NH₂, or —NO₂; and
R₅, R₆ and R₇ are independently selected from H, halogen, C₁-C₃ alkyl, C₁-C₃ perfluoroalkyl, —O—C₁-C₃ perfluoroalkyl, C₁-C₃ alkoxy, —OH, —NH₂, or —NO₂, provided that at least one of R₅, R₆ and R₇ is not H.

6. The composition of claim 1, wherein the compound of formula (I) is a compound of formula (VI), or a pharmaceutically acceptable salt, solvate or ester thereof: 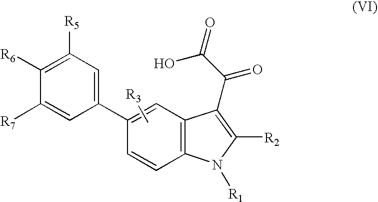
wherein:
R₁ is selected from benzyl, the benzyl group being optionally substituted by from 1 to 3 groups independently selected from halogen, C₁-C₄ alkyl, C₁-C₃ perfluoroalkyl, —O—C₁-C₃ perfluoroalkyl, and C₁-C₃ alkoxy;
R₂ is H;
R₃ is H; and
R₅, R₆ and R₇ are independently selected from H, halogen, C₁-C₃ alkyl, C₁-C₃ perfluoroalkyl, —O—C₁-C₃ perfluoroalkyl and C₁-C₃ alkoxy, provided that at least one of R₅, R₆ and R₇ is not H.

7. The composition of claim 1, wherein the compound of formula (I) is:
a) {1-Methyl-6-[4-(trifluoromethoxy)phenyl]-1H-indol-3-yl}(oxo)acetic acid;
b) {1-Methyl-6-[4-(trifluoromethyl)phenyl]-1H-indol-3-yl}(oxo)acetic acid;
c) {1-Ethyl-6-[4-(trifluoromethoxy)phenyl]-1H-indol-3-yl}(oxo)acetic acid;
d) {1-Ethyl-6-[4-(trifluoromethyl)phenyl]-1H-indol-3-yl}(oxo)acetic acid;
e) {1-Benzyl-6-[4-(trifluoromethoxy)phenyl]-1H-indol-3-yl}(oxo)acetic acid;
f) {1-Benzyl-6-[4-(trifluoromethyl)phenyl]-1H-indol-3-yl}(oxo)acetic acid;
g) {1-[4-(tert-Butyl)benzyl]-6-[4-(trifluoromethyl)phenyl]-1H-indol-3-yl}(oxo)acetic acid;
h) {1-[4-(tert-Butyl)benzyl]-6-[4-(trifluoromethoxy)phenyl]-1H-indol-3-yl}(oxo)acetic acid;
i) {1-Benzyl-5-[4-(trifluoromethyl)phenyl]-1H-indol-3-yl}(oxo)acetic acid;
j) {6-[4-(tert-Butyl)phenyl]-1-methyl-1H-indol-3-yl}(oxo)acetic acid;
k) [5-(4-Acetylphenyl)-1-benzyl-1H-indol-3-yl](oxo)acetic acid;
l) {1-Benzyl-5-[4-(trifluoromethoxy)phenyl]-1H-indol-3-yl}(oxo)acetic acid;
m) {1-Benzyl-4-[4-(trifluoromethyl)phenyl]-1H-indol-3-yl}(oxo)acetic acid;
n) {1-Benzyl-5-[4-(tert-butyl)phenyl]-1H-indol-3-yl}(oxo)acetic acid;
o) [1-Benzyl-5-(3-chloro-4-fluorophenyl)-1H-indol-3-yl](oxo)acetic acid;
p) {1-Benzyl-5-[3,5-bis(trifluoromethyl)phenyl]-1H-indol-3-yl}(oxo)acetic acid;
q) {1-Benzyl-7-[4-(trifluoromethoxy)phenyl]-1H-indol-3-yl}(oxo)acetic acid;
r) [1-Benzyl-7-(3-chloro-4-fluorophenyl)-1H-indol-3-yl](oxo)acetic acid;
s) {1-(4-tert-Butylbenzyl)-5-[4-(trifluoromethoxy)phenyl]-1H-indol-3-yl}(oxo)acetic acid;
t) {1-Benzyl-4-[4-(trifluoromethoxy)phenyl]-1H-indol-3-yl}(oxo)acetic acid;
u) [1-Benzyl-6-(3-chlorophenyl)-1H-indol-3-yl](oxo)acetic acid;
v) {1-Benzyl-5-[3-(trifluoromethoxy)phenyl]-1H-indol-3-yl}(oxo)acetic acid;
w) {1-(4-Methylbenzyl)-5-[4-(trifluoromethoxy)phenyl]-1H-indol-3-yl}(oxo)acetic acid;
x) {1-(4-Fluorobenzyl)-5-[4-(trifluoromethoxy)phenyl]-1H-indol-3-yl}(oxo)acetic acid;
y) [1-Butyl-5-(4-chlorophenyl)-1H-indol-3-yl](oxo)acetic acid;
z) [1-Butyl-5-(3-chlorophenyl)-1H-indol-3-yl](oxo)acetic acid;
aa) [1-Butyl-5-(3-methoxyphenyl)-1H-indol-3-yl](oxo)acetic acid;
bb) [1-Butyl-5-(4-methoxyphenyl)-1H-indol-3-yl](oxo)acetic acid;
cc) {1-Butyl-5-[4-(trifluoromethyl)phenyl]-1H-indol-3-yl}(oxo)acetic acid;
dd) [1-(4-tert-Butylbenzyl)-5-(3-methylphenyl)-1H-indol-3-yl](oxo)acetic acid;
ee) [1-(4-tert-Butylbenzyl)-5-(3-methoxyphenyl)-1H-indol-3-yl](oxo)acetic acid;
ff) [1-(4-tert-Butylbenzyl)-5-(4-tert-butylphenyl)-1H-indol-3-yl](oxo)acetic acid;
gg) [1-(4-tert-Butylbenzyl)-5-(3-chlorophenyl)-1H-indol-3-yl](oxo)acetic acid;
hh) [1-(4-tert-Butylbenzyl)-5-(4-chlorophenyl)-1H-indol-3-yl](oxo)acetic acid;
ii) [1-(4-tert-Butylbenzyl)-5-(2-methylphenyl)-1H-indol-3-yl](oxo)acetic acid;
jj) {1-(2-Ethylbutyl)-5-[4-(trifluoromethoxy)phenyl]-1H-indol-3-yl}(oxo)acetic acid;
kk) {2-[(Acetyloxy)methyl]-1-(4-methylbenzyl)-5-[4-(trifluoromethoxy)phenyl]-1H-indol-3-yl}(oxo)acetic acid;
ll) {2-(Hydroxymethyl)-1-(4-methylbenzyl)-5-[4-(trifluoromethoxy)phenyl]-1H-indol-3-yl}(oxo)acetic acid;
mm) {2-[(Acetyloxy)methyl]-1-benzyl-5-[4-(trifluoromethoxy)phenyl]-1H-indol-3-yl}(oxo)acetic acid;
nn) {1-Benzyl-2-(hydroxymethyl)-5-[4-(trifluoromethoxy)phenyl]-1H-indol-3-yl}(oxo)acetic acid;
oo) [5-(3-Chlorophenyl)-1-cyclopentyl-1H-indol-3-yl]-oxo-acetic acid;
pp) [5-(3-chlorophenyl)-1-(cyclobutylmethyl)-1H-indol-3-yl](oxo)acetic acid;
qq) [5-(3-chlorophenyl)-1-(3-methylcyclopropyl)-1H-indol-3-yl](oxo)acetic acid;
rr) [5-(3-chlorophenyl)-1-(cyclohexylmethyl)-1H-indol-3-yl](oxo)acetic acid;
ss) 5-(4-trifluoromethylphenyl)-1-(cyclopentyl)-1H-indol-3-yl](oxo)acetic acid;
tt) [5-(4-trifluoromethylphenyl)-1-(cyclobutylmethyl)-1H-indol-3-yl](oxo)acetic acid;
uu) [5-(4-trifluoromethylphenyl)-1-(3-methylcyclopentyl)-1H-indol-3-yl](oxo)acetic acid;
vv) [5-(4-trifluoromethylphenyl)-1-(cyclohexylmethyl)-1H-indol-3-yl](oxo)acetic acid;
ww) [5-(4-trifluoromethylphenyl)-1-(cyclopentylpropyl)-1H-indol-3-yl](oxo)acetic acid;
xx) [5-(3-trifluoromethylphenyl)-1-(cyclopentyl)-1H-indol-3-yl](oxo)acetic acid;
yy) [5-(3-trifluoromethylphenyl)-1-(cyclobutylmethyl)-1H-indol-3-yl](oxo)acetic acid;
zz) [5-(3-trifluoromethylphenyl)-1-(3-methylcyclopentyl)-1H-indol-3-yl](oxo)acetic acid;
aaa) [5-(3-trifluoromethylphenyl)-1-(cyclohexylmethyl)-1H-indol-3-yl](oxo)acetic acid;
bbb) [5-(3-trifluoromethylphenyl)-1-(cyclopentylpropyl)-1H-indol-3-yl](oxo)acetic acid;
ccc) [5-(4-methoxyphenyl)-1-(cyclohexylmethyl)-1H-indol-3-yl](oxo)acetic acid; or
or a pharmaceutically acceptable salt or solvate or ester thereof.

8. The composition of claim 1, wherein the compound of formula (I) is [1-(4-tert-Butylbenzyl)-5-(3-methylphenyl)-1H-indol-3-yl](oxo)acetic acid, or a pharmaceutically acceptable salt, solvate or ester thereof.

9. The composition of claim 3, wherein the composition comprises:
(a) 0.01% to 30% w/w of a compound of formula I or a pharmaceutically acceptable salt, solvate or ester thereof;
(b) 25% to 50% w/w of a solubilizer or emulsifier A; and
(c) 25% to 50% w/w of a solubilizer or emulsifier B.

10. The composition of claim 3, wherein the composition comprises:
(a) 0.01% to 30% w/w of a compound of formula I or a pharmaceutically acceptable salt, solvate or ester thereof;
(b) 25% to 50% w/w of a solubilizer or emulsifier A; and
(c) 25% to 50% w/w of a solubilizer or emulsifier C.

11. The composition of claim 3, wherein the composition comprises:
(a) 0.01% to 30% w/w of a compound of formula I or a pharmaceutically acceptable salt, solvate or ester thereof;
(b) 25% to 50% w/w of a solubilizer or emulsifier A; and
(c) 25% to 50% w/w of a solubilizer or emulsifier D.

12. The composition of claim 3, wherein the composition comprises:
(a) 0.01% to 30% w/w of a compound of formula I or a pharmaceutically acceptable salt, solvate or ester thereof;
(b) 25% to 50% w/w of a solubilizer or emulsifier A;
(c) 25% to 50% w/w of a solubilizer or emulsifier B; and
(d) 5% to 35% w/w of a solubilizer or emulsifier D.

13. The composition of claim 3, wherein the composition comprises:
(a) 0.01% to 30% w/w of a compound of formula I or a pharmaceutically acceptable salt, solvate or ester thereof;
(b) 25% to 50% w/w of a solubilizer or emulsifier A;
(c) 25% to 50% w/w of a solubilizer or emulsifier C; and
(d) 5% to 35% w/w of a solubilizer or emulsifier D.

14. The composition of claim 3, wherein the composition comprises:
(a) 0.01% to 30% w/w of a compound of formula I or a pharmaceutically acceptable salt, solvate or ester thereof;
(b) 25% to 50% w/w of a w/w of a solubilizer or emulsifier C;
(c) 25% to 50% w/w of a solubilizer or emulsifier B; and
(d) 5% to 35% w/w of a solubilizer or emulsifier D.

15. The composition of claim 3, wherein the composition comprises:
(a) 0.01% to 20% w/w of a compound of formula I or a pharmaceutically acceptable salt, solvate or ester thereof;
(b) 30% to 45% w/w of a solubilizer or emulsifier A;
(c) 30% to 45% w/w of a solubilizer or emulsifier B; and
(d) 10% to 28% w/w of a solubilizer or emulsifier D.

16. The composition of claim 3, wherein the composition comprises:
(a) 0.01% to 20% w/w of a compound of formula I or a pharmaceutically acceptable salt, solvate or ester thereof;
(b) 30% to 45% w/w of a solubilizer or emulsifier A;
(c) 30% to 45% w/w of a solubilizer or emulsifier C; and
(d) 10% to 28% w/w of a solubilizer or emulsifier D.

17. The composition of claim 3, wherein the composition comprises:
(a) 0.01% to 20% w/w of a compound of formula I or a pharmaceutically acceptable salt, solvate or ester thereof;
(b) 30% to 45% w/w of a solubilizer or emulsifier C;
(c) 30% to 45% w/w of a solubilizer or emulsifier B; and
(d) 10% to 28% w/w of a solubilizer or emulsifier D.

18. The composition of claim 3, wherein the composition comprises:
(a) 0.01% to 10% w/w of a compound of formula I or a pharmaceutically acceptable salt, solvate or ester thereof;
(b) 36% to 40% w/w of a solubilizer or emulsifier A;
(c) 36% to 40% w/w of a solubilizer or emulsifier B; and
(d) 15% to 25% w/w of a solubilizer or emulsifier D.

19. The composition of claim 3, wherein the composition comprises:
(a) about 10% w/w of a compound of formula I or a pharmaceutically acceptable salt, solvate or ester thereof;
(b) about 36% w/w of a solubilizer or emulsifier A;
(c) about 36% w/w of a solubilizer or emulsifier B; and
(d) about 18% w/w of a solubilizer or emulsifier D.

20. The composition of claim 3, wherein the composition comprises:
(a) about 20% w/w of a compound of formula I or a pharmaceutically acceptable salt, solvate or ester thereof;
(b) about 32% w/w of a solubilizer or emulsifier A;
(c) about 32% w/w of a solubilizer or emulsifier B; and
(d) about 16% w/w of a solubilizer or emulsifier D.

21. The composition of claim 3, wherein the composition comprises:
(a) about 20% w/w of a compound of formula I or a pharmaceutically acceptable salt, solvate or ester thereof;
(b) about 47% w/w of a solubilizer or emulsifier D;
(c) about 14% w/w of another solubilizer or emulsifier D;
(d) about 14% w/w of yet another solubilizer or emulsifier D; and
(e) about 5% w/w of a solubilizer or emulsifier C.

22. The composition of claim 3, wherein the composition comprises:
(a) about 6% w/w of a compound of formula I or a pharmaceutically acceptable salt, solvate or ester thereof;
(b) about 2% w/w of a solubilizer or emulsifier C;
(c) about 0.5% of a thickening agent;
(d) about 0.4% of a pH modifier; and
(e) about 91% of water.

23. The composition according to claim 3, wherein said solubilizer or emulsifier A is Cremophor EL, Solutol HS-15 or Polysorbate 80; said solubilizer or emulsifier B is Solutol HS-15, Polysorbate 80 or Cremophor EL; said solubilizer or emulsifier C is Polysorbate 80, Cremophor EL or Solutol HS-15; said solubilizer or emulsifier D is Capryol 90, Phosal 53 MCT, Lauroglycol, Capmul MCM, Capmul PG-8, Captex 355, Labrasol or Propylene Carbonate.

24. A method of treating Alzheimer's disease comprising the administration of the composition of claim 3 to a mammal in need thereof.

25. A method of increasing or normalizing levels of plasmin concentration comprising the administration of the composition of claim 3 to a mammal in need thereof.

26. The method of claim 24 where the mammal is a human.

27. The method of claim 25 where the mammal is a human.
